(19) United States
(12) Patent Application Publication
(10) Pub. No.: US 2015/0110852 A1
(43) Pub. Date: Apr. 23, 2015

(54) SHEET AND LIQUID COMBINATION SYSTEMS FOR DERMAL DELIVERY OF LIDOCAINE, DICLOFENAC, AND OTHER DRUGS

(71) Applicant: Jie Zhang, Salt Lake City, UT (US)

(72) Inventor: Jie Zhang, Salt Lake City, UT (US)

(21) Appl. No.: 14/399,834

(22) PCT Filed: May 8, 2013
(86) PCT No.: PCT/US13/40146

§ 371 Date: Nov. 7, 2014

Related U.S. Application Data
(60) Provisional application No. 61/644,362, filed on May 8, 2012.

(51) Int. Cl. A61K 9/70; A61K 31/192; A61K 31/196; A61K 31/167; A61K 31/445

(52) U.S. Cl. CPC A61K 9/703; A61K 31/167; A61K 31/445; A61K 31/196; A61K 31/192
USPC 424/449; 514/626; 514/330; 514/567; 514/570; 604/113

(57) ABSTRACT

A system for dermal delivery of lidocaine, bupivacaine, diclofenac, or ketoprofen is provided which comprises at least two components, for example, a sheet of a solid and flexible material, and a vehicle liquid comprising a solvent and optionally other ingredients. A drug, selected from group consisting of lidocaine, bupivacaine, diclofenac, and ketoprofen, can be impregnated in the sheet or contained in the vehicle liquid. These two components can be stored separately and joined either shortly before or at the time of application. To use the system, the vehicle liquid may be applied either on the target skin area or on the sheet, and the sheet may then be applied on the target skin area so that the vehicle liquid is positioned between the sheet and the skin and brought into contact with the ingredients impregnated in the sheet.

### RELATED APPLICATIONS

This application claims priority to U.S. Provisional Application No. 61/644,362 filed May 8, 2012, entitled “SHEET AND LIQUID COMBINATION SYSTEMS FOR DERMAL DELIVERY OF LIDOCAINE, DICLOFENAC, AND OTHER DRUGS,” which is incorporated herein by reference in its entirety.

### BACKGROUND

Dermal drug delivery systems (comprising drug formulations applied on the skin or mucosa) are widely used in treating medical conditions not only in the “surface tissues” such as skin or mucosa but also in deeper tissues such as musculoskeletal tissues. One of the common dosage forms for dermal drug delivery is dermal patches of various types. A reservoir patch typically comprises a thin bag (reservoir) containing the drug solution. The solvent in the solution and the adhesive layer (between the solution and the skin during application) usually serve as the vehicle for the delivery of the drug into the skin. A drug-in-adhesive patch typically uses the adhesive as the vehicle for the delivery of the drug. A hydrogel based patch, such as the Lidoderm Patch, typically uses the hydrogel as the vehicle for the drug delivery. So in existing dermal drug delivery patches, the vehicle for the drug delivery is contained in the patch. While that is convenient for the user, it has several shortcomings. In a reservoir patch, the solvent must be contained in a thin reservoir bag, which means the patch is bulky and cannot be cut for covering target skin areas with different or irregular sizes. In a hydrogel based patch, the hydrogel has to have certain properties such as being in a particular pH window and being sufficiently adhesive and cohesive, which can prevent the formulation from having the optimal properties for delivering the drug into the skin. In a drug-in-adhesive patch, the thin adhesive layer has to serve both as means to hold the patch on the skin and as the vehicle for drug delivery. The fulfillment of the first task often compromises the second one, which can mean sub-optimal drug delivery.

Another class of commonly used dermal drug delivery systems include topical solutions, gels, creams, and ointments. These liquid or semisolid dosage forms are typically applied to the skin surface without occlusion, which leads to quick evaporation of the solvent or unintentional removal of the formulation by objects such as clothing. When the solvent vehicle is evaporated or the formulation is removed from skin surface, the drug delivery significantly slows or even stops, resulting in short effective delivery time and low delivery quantity. As a result, many liquid and semisolid drug formulations have to be applied multiple times a day.

Therefore, for some dermal drug delivery applications, it is desirable to have a class of new dosage forms that can address the aforementioned shortcomings of the patch and liquid/semisolid dosage forms.

### DETAILED DESCRIPTION

In some embodiments of the current invention, the dermal drug delivery system comprises two components. The first component comprises a sheet of a solid and flexible material with a certain barrier property, and the second component comprises a liquid comprising a solvent. The first and the second components both may optionally further comprise other ingredients. An active drug can be impregnated in the sheet or contained in the liquid. These two components are stored separately and joined shortly before or at the time of application. This system is generally referred to as the Sheet and Liquid Combination system hereafter. Certain aspects of Sheet and Liquid Combination system technology are described in PCT/US2011/059813, which is incorporated herein by reference in its entirety.

To use the system, the liquid is applied either on the target skin area or on the sheet, and the sheet is then applied on the target skin area so that the liquid is between the sheet and the skin, absorbed into the solid sheet in whole or in part, or at least partially absorbed into the sheet and partially present between the sheet and the skin. After the sheet and the liquid are combined in this pre-designed way, the ingredients impregnated in the sheet and in the liquid are combined to form a combined (new) formulation that is capable of delivering the drug through the skin at the desired rates. The combined formulation can also provide the adhesion needed to affix the sheet on the skin.

At least one of the two aforementioned components comprises a drug (active ingredient). In many embodiments, the drug is contained in the sheet. In some of the embodiments, each of the two aforementioned components alone is not able to deliver the drug at the desired rate, but the combination is. In other embodiments, one of the two components alone may be able to deliver the drug at a desired rate, but the other component can provide some desirable properties. For instance, a low viscosity drug solution containing a drug may be able to deliver the drug into the skin at a desired rate if it is kept on the skin for long enough time. However, it is difficult to keep a low viscosity solution on skin for long time. Therefore, a system comprising a low viscosity drug solution as the liquid component and a sheet with a liquid retention layer and a barrier film (discussed in further detail below) as the sheet component may be used to conveniently keep the drug solution on the skin for longer time to achieve longer effective drug delivery time.

It is noted that, as used in this specification and the appended claims, singular forms of “a,” “an,” and “the” include plural referents unless the content clearly dictates otherwise.

All percentage values in this disclosure are weight percentages unless specifically expressed otherwise.

“Vehicle liquid”, or “liquid” in the Sheet and Liquid Combination system, means a liquid comprising a vehicle solvent system that is used to transdermally deliver the drug at rates high enough to achieve the desired effect(s). Vehicle liquid can be a free flowing liquid, a viscous liquid, a gel or cream or any semisolid that cannot flow but can be spread into a layer, a liquid soaked in an absorbent sheet, a water-containing foam, or liquid in a solidified gel such as a hydrogel. Vehicle liquid may comprise only a single ingredient such as water, or multiple ingredients such as water, thickening agents, adhesion agents, etc. The vehicle liquid may also contain an active drug, which may be dissolved or suspended in the liquid.

A “layer of vehicle liquid” means a continuous layer of the vehicle liquid, a substantially two dimensional presence but not necessarily continuous layer of the vehicle liquid (e.g., densely populated fine droplets of the vehicle liquid), or the vehicle liquid existing in a substantially two dimensional sheet of material (e.g. absorbed into a sheet of material to form a “wet” sheet).

“Sheet,” as used in describing the Sheet and Liquid Combination system of the current invention, means a sheet of solid material such as a paper, film, tape, fabric, sponge, or a combination thereof. The sheet can have a barrier film layer such as a polymer film or tape. The sheet can have a moisture vapor transfer rate (MVTR) that is very low, so that it allows little evaporation of the solvent in the vehicle liquid placed between it and the skin during the application period. Alternatively, the sheet can have a pre-determined MVTR that allows the solvent in the vehicle liquid to evaporate through it at rates such that sufficient amount of solvent in the vehicle liquid placed between it and the skin stays long enough to deliver the desired amount of the drug, but by the end of the application, enough of the water has evaporated so that there is minimal or no residue water left on the skin. This pre-determined MVTR is defined as “dry-ending MVTR”.

In many embodiments of the Sheet and Liquid Combination systems of the current invention, the sheet is not adhesive to dry human skin without a liquid. In those embodiments, even if impregnated with an adhesive agent, the sheet itself is not adhesive to dry human skin but can become adhesive to dry human skin when combined with the liquid part. For example, all the sheets in the Examples of the current disclosure are not adhesive to dry human skin without the liquid part.

In some of the embodiments of the current invention, once the sheet and the vehicle liquid are joined and applied on the skin, the solvent in the vehicle liquid is present long enough to deliver a clinically sufficient amount of the drug into the skin. In such embodiments, the sheet's MVTR must be low enough to keep the solvent present long enough to deliver the said amount of the drug.

“MVTR” means moisture vapor transfer rate, as measured with the method used by 3M Co. to measure the MVTR values of a number of tapes published on page 33, Medical Single Coated Film Tapes Selection Guide, Version 5.0 October 2008, or similar methods. When a sheet is said to have a certain MVTR value, it means at least some part of the sheet has that MVTR value, and potentially that much or most of the sheet's area has that MVTR value, but it does not necessarily mean that the entire sheet area has that MVTR value.

In some embodiments of the current invention, the sheet has a “lateral diffusion function” or comprises a “lateral diffusion layer”. The lateral diffusion layer is typically a layer of material very absorbent to the vehicle liquid, such as water. When a droplet of the vehicle liquid is placed on the lateral diffusion layer, it is quickly absorbed into the layer and spread laterally to cover an area much larger than the initial size, as measured by cross-sectional area, of the droplet. (When a drop of water is placed on a Kleenex tissue, it will quickly spread laterally into a circle whose diameter is much larger than the diameter of the initial water drop. However, a drop of water placed on an aluminum foil will not diffuse much laterally. In this comparison, the Kleenex tissue has the lateral diffusion function and it or a material with similar lateral diffusion function may be used as a lateral diffusion layer in a sheet, while the aluminum foil does not have the lateral diffusion function and would not work well as a lateral diffusion layer in a sheet). The lateral diffusion layer is typically the layer in the sheet that is in contact with the vehicle liquid when the combination of the sheet and the vehicle liquid is applied on the skin. The lateral diffusion layer functions to cause the vehicle liquid to spread fairly evenly over the desired area under the sheet, even if the initial application of the vehicle liquid on the skin or the sheet is not very even. For example, water as the vehicle liquid can be sprayed on the skin to cover the target skin area with densely populated water beads, but not quite a continuous layer of water. The lateral diffusion layer applied over the water beads will quickly absorb the water beads. The absorbed water will then quickly spread laterally, so that the entire sheet area will have even water distribution. Many absorbent materials may be used as the material for the lateral diffusion layer, including gauze (woven or non-woven), paper, foam (especially open-cell foam), cloth, and other fabric materials. For example, the fabric (gauze) layer in the fabric-tape laminate sheets in many of the following Examples functions as a lateral diffusion layer.

Unless specified otherwise, when a tape-fabric or film-fabric laminate sheet is said to be “applied to the skin,” it means the sheet is applied in such a way that the fabric side of the laminate is the side that is in direct contact with the skin and the applied vehicle liquid.

Since the lateral diffusion layer is absorbent to the vehicle liquid, it typically has very high MVTR (moisture vapor transfer rate). Therefore, in order to maintain water or other solvent in the vehicle liquid between the sheet and skin for sufficient time to deliver the desired amount of the drug into the skin, the lateral diffusion layer is often laminated with a “MVTR control layer” to form a sheet that has both the lateral diffusion function and proper MVTR. Typically, the “MVTR control layer” is a layer of material that has much lower MVTR than that of the lateral diffusion layer and thus dominantly determines the overall MVTR for the sheet. The MVTR control layer is typically a layer of plastic film or tape with desired MVTR. The desired MVTR for a given sheet-liquid combination dermal drug delivery system depends on the drug delivery requirements. If the drug is to be continuously delivered for a long time, such as 8-12 hours, the MVTR should be low, such as lower than 800 g/m²/24 hour, or lower than 200 g/m²/24 hour, so that sufficient amount of the solvent is present to serve as the vehicle for delivering the drug for most or all of the system's application time. If the drug delivery period is short, such as one hour, the MVTR can be low, such as lower than 200 g/m²/24 hour, but can also be higher, such as about 800 g/m²/24 hour, so that the solvent stays in the system for long enough time to deliver sufficient amount of the drug, but at the end of the application period, most of the liquid has evaporated and not much residual is left on the skin after the system is removed from the skin.

The MVTR control layer can comprise a barrier film with adequate MVTR. In the current invention, the phrase “barrier film” means a film with MVTR lower than 2,000 g/m²/24 hour, and in some instances even lower than about 100 g/m²/24 hour. Many tapes used in the current invention, such as 3M 9832, 3M 9834, and 9830 tapes, comprise a barrier film layer. A tape is typically a film coated with a layer of adhesive.

The fabric (lateral diffusion or liquid retention) layer and the MVTR control layer can be conveniently laminated together by using a layer of adhesive. For example, 3M9832 tape is a polyurethane film with one side coated with a layer of adhesive. As shown in some of the examples below, a layer of fabric can be placed on the adhesive side of the tape to form a fabric/barrier film laminate. Alternatively, in some of the embodiments of the current invention, the fabric is laminated to the barrier film (MVTR control layer) by heat.

“Fabric layer” or “fabric” means a material or a layer of material that is absorbent of water or water based solution, including woven and non-woven materials. For example, a layer of non-woven rayon-polyester blend material as that used in some of the examples below is a fabric layer. In contrast, wax-coated paper is not “fabric” by the definition herein because it is not absorbent to water. A fabric layer can serve as the liquid retention layer (fluid retention layer) and the lateral diffusion layer. The fabric liquid retention layer in a sheet of the current intention can help keep more liquid between the sheet and the skin surface (for example, compared to a sheet with just a barrier film and no fabric layer). Being able to hold larger volume of the liquid can be especially important for delivering drugs that require long delivery time so the unavoidable loss of solvent to skin absorption and evaporation does not reduce the amount of the solvent to the point that can negatively affect the drug delivery.

In some of the embodiments, the sheet in the Sheet and Liquid Combination system can have a liquid retention layer for keeping the sufficient amount of the liquid available to the skin for long enough time to deliver the desired amount of the drug. A liquid retention layer, such as a fabric layer in a fabric-barrier film laminate sheet, can absorb the vehicle fluid and keep it relatively evenly available to the skin. A liquid retention layer can have the lateral diffusion function and can be the lateral diffusion layer at the same time. With a liquid retention layer, the sheet can keep more liquid between it and the skin during the application of the Sheet and Liquid Combination system.

“Fastening agent” means a substance that “fastens” a drug or an excipient on the sheet. Without the fastening agent, the drug or the excipient impregnated in the sheet may be only loosely held by the sheet and can be unintentionally removed from the sheet when the sheet is shaken, bent, touched, or rubbed. Substances that can bind with both the sheet and the drug or excipient can function as fastening agents. Such substances include but are not limited to polyvinylpyrrolidone (PVP), poly vinyl alcohol (PVA), ethyl cellulose, hydroxy propyl cellulose, carrageenan, and gum Arabic.

“Adhesion agent” is a substance capable of facilitating the adhesion between the skin and the sheet. It can initially exist in the vehicle liquid. It can also initially exist in the sheet, and dissolve into the vehicle liquid when the vehicle liquid and the sheet are brought into contact. Substances that are soluble in the vehicle liquid and increase the vehicle liquid's adhesion to skin or to the sheet can be used as adhesion agents. Such substances include, but are not limited to, PVP, PVA, poly acrylic polymers such as the Carbomer polymers marketed by Noveon (e.g Carbopol 981), xantham gum, and gum Arabic. Adhesion agent can also be a combination of two or more substances. For example, polyvinylpyrrolidone-glycerin mixture and polyvinylpyrrolidone-poly ethylene glycol 400 mixture, with appropriate polyvinylpyrrolidone percentages, can be used as adhesion agents.

“Normal human skin” means human skin with an intact stratum corneum layer and normal skin temperature (typically in the range of 30-36° C.). Normal human skin can include skin that is suffering from a disease or pain but has an intact stratum corneum layer.

“Normal ambient conditions” means temperatures in the range of 20-35° C. and relative humidity in the range of 0 to 80%.

The phrases “anesthesia in skin”, “anesthetized skin”, “numbness”, and the like, mean the skin is anesthetized at least to the extent that it feels numb when it is scratched or poked with the end of a straightened paper clip. This skin numbing effect can be observed in a single human subject or can be observed in a formal clinical trial. Since there are often outliers in human testing, in the case of a formal clinical trial or trials, this term is used to denote that at least 70% of the subjects in a group of at least 24 subjects have the effect. Alternatively, the definition can be that the effect is statistically significant according to the U.S. Food and Drug Administration's definition at the time of testing.

The term “free of water” when used to describe a sheet or a component of a sheet means that the quantity or concentration of water in said sheet or component of sheet is so low that the sheet or the component of the sheet is dry to the touch and cannot deliver the drug at the desired rate without additional water. “Free of water” does not necessarily mean there is absolutely no water.

The term “appropriate quantity” when referring to the quantity of the vehicle liquid applied on the skin or sheet means a quantity of the vehicle liquid that is high enough and can last long enough to allow a sufficient amount of the drug to be delivered transdermally into the skin to achieve the desired clinical effect(s), but not so high as to cause problems such as overflow or running. The “appropriate quantity” can depend on the MVTR of the particular sheet, viscosity of the liquid, among other factors, and can be in the range of 2 to 400 milligrams per cm² (mg/cm²), including the range of 5 to 200 mg/cm², and including the range of 10 to 100 mg/cm².

The phrase “between the skin and the sheet”, and the like, when referring to the position of the vehicle liquid relative to the skin and the sheet means the vehicle liquid is between the skin and the sheet and includes situations in which the vehicle liquid is applied onto or absorbed into the sheet, or partially absorbed into the sheet and partially present between the sheet and the skin, and the sheet is applied on the skin.

“Target skin area” in general means an area of human (or other mammal) skin into which the delivery of the drug is expected to produce the desired clinical effect(s). For anesthetizing the skin before painful procedures, reducing the pain associated with shingles, and other pain associated with diseases or trauma of the skin, the target skin area can be the skin area suffering from the pain. For treating musculoskeletal pain, the target skin area can be the skin area under or adjacent to which the musculoskeletal pain exists. Target skin area can also be the skin area over a “trigger point”, a hyperirritable spot in the tissue (usually muscle tissue) that sometimes can cause pain quite distant from the trigger point itself. Target skin area can also be a skin area over a tissue into which physicians would inject a local anesthetic or other drugs to reduce pain (e.g., skin area over the site into which physicians inject lidocaine to reduce shoulder pain). Target skin area can be an area of diseased or normal skin.

The term “properly adhered”, “proper adhesion”, and the like, when referring to the adhesion of the sheet on human skin, means that the adhesion is such that the sheet can stay on a normal human skin area under normal ambient conditions for at least 15 minutes regardless of how the skin area is positioned (e.g., face up, face down, or at an angle such that the sheet is vertical to the ground).

“Pain reduction” can mean the reduction of pain sensed by a human being in general. Pain reduction can also mean statistically significant reduction of pain as measured by methods commonly used in clinical trials employing commonly used patient selection criteria and test conditions. Such methods include, without limitation, the visual analog pain scale method.

Many drugs can be delivered using the systems and methods of the current invention. These drugs include, but are not limited to, local anesthetic drugs such as lidocaine, tetracaine, prilocalne, bupivacaine, benzocaine, ropivacaine, etidocaine, mepivacaine, dibucaine; non-steroidal anti-inflammatory drugs (NSAIDs) such as diclofenac and ketoprofen; capsaicin; drugs that are used to treat neuropathic pain such as N-methyl-D-asparate (NMDA) receptor antagonists (e.g. gabapentin) and ketamine. In some systems of the invention, the drugs which particularly benefit from the systems and methods of the current invention are those which are subject to hydrolytic degradation when in contact with water.

All drugs used in the embodiments of the current invention can be in their free base or free acid form or any salt form. For example, “lidocaine” as used in the current invention means lidocaine base or any salt of lidocaine such as lidocaine hydrochloride. Likewise, “diclofenac” means diclofenac or any of its salts such as diclofenac sodium and diclofenac epolamine.

In some embodiments of the current invention, a system for delivering a local anesthetic agent, such as lidocaine or bupivacaine, into human skin comprises a first component and a second component. The first component comprises a sheet of a solid and flexible material and is impregnated with a local anesthetic agent. The sheet can also be free of water. The second component comprises a liquid comprising water. To use the system to deliver the local anesthetic into human skin or skin of another mammal, the liquid is applied either on the target skin area or on the sheet, and the sheet is then applied on the target skin area, so that the liquid is between the sheet and the skin. Once the liquid is in contact with the sheet and the skin, the local anesthetic impregnated in the sheet begins to dissolve into the liquid and is delivered into the skin via the liquid. The sheet is constructed to have a low enough MVTR to keep the water in the liquid between the sheet and the skin for a time sufficient to deliver the needed amount of the drug. The system is maintained in place for a time sufficient to deliver a sufficient amount of the local anesthetic agent to achieve the desired pain reduction or anesthetic effect. An adhesion agent can be incorporated in the vehicle liquid to enable the vehicle liquid to serve as a weak “glue” to keep the sheet properly adhered on the skin for the duration of the application period. The adhesion can be strong enough so that the sheet stays on the skin even if the patient moves or changes the position of the body part that contains the skin area. Alternatively, the adhesion agent can be impregnated in the sheet, and dissolves into the liquid when liquid is brought into contact with the sheet. The liquid can be applied on the target skin area or the sheet in several different ways, including sprayed on the skin or the sheet, brushed on the skin or the sheet; soaked in a sheet of absorbent material (to form a “wet sheet”) which is then applied on the skin or the sheet, or crosslinked into a solidified hydrogel sheet which is then applied on the skin or the sheet. Optionally, the sheet can have a dry-ending MVTR so that it can keep the water in the vehicle liquid under the sheet long enough to deliver a sufficient amount of the drug into the skin to achieve a desired anesthetic or pain reduction effect, but by the end of the application period a sufficient amount of water in the vehicle liquid under the sheet has evaporated through the sheet so that when the sheet is removed from the skin at the end of the application period, the skin is substantially free of liquid so that the need to wipe off the residual liquid on the skin is avoided.

The liquid can have an appropriate viscosity for facilitating the application on the skin or the sheet and the delivery of the drug into the skin. For vehicle liquid that is to be spread on the skin, if the viscosity is too low, maintaining an appropriate quantity of the vehicle liquid on the skin before the sheet is applied can be difficult because a low viscosity liquid can flow away from the target skin area easily. If the viscosity is too high, the application of it on the target skin area can be difficult. Therefore, the viscosity should not be too high or too low, and can be in the range of 100 to 1 million centipoises, alternatively in the range of 500 to 200,000 centipoise, and alternatively in the range of 1,500 to 50,000 centipoise. However, in systems in which the vehicle liquid is to be sprayed on the sheet or skin, the viscosity of the vehicle liquid is preferably lower, and can be as low as that of water or lower.

The drug can be impregnated in the sheet with the help of a “fastening agent”. For example, lidocaine and poly vinyl alcohol (PVA) can be dissolved in an isopropyl alcohol:water solution (e.g. 50:50 by weight), which is then evenly dispensed into the absorbent fabric part of a sheet. After evaporating off the isopropyl alcohol and water, the PVA solid binds with both the fabric and the lidocaine, thus “fastening” the lidocaine on the sheet.

Other embodiments of the current invention are related to a system comprising a liquid comprising water and a sheet comprising a barrier film (whose MVTR is lower than 1,000/m²/24 hour) and lidocaine, wherein said lidocaine is distributed co-extensively with said barrier film (either in the barrier film or in another layer of material co-extensive with the barrier film), and wherein said liquid is brought into contact with said lidocaine within one hour of application of said system on a mammal's skin.

In some embodiments of the current invention, a system for delivering a local anesthetic agent, such as lidocaine or bupivacaine, into human skin comprises a first component and a second component. The first component comprises a sheet comprising a barrier film and can be free of water. The second component comprises a liquid comprising water and a local anesthetic agent (e.g., lidocaine at a concentration not higher than about 4%, or even not higher than about 2.5%). An adhesion agent can be present in the sheet or the liquid. To use the system to deliver the local anesthetic into human skin or skin of another mammal, the liquid is applied either on the target skin area or on the sheet, and the sheet is then applied on the target skin area, so that the liquid is between the sheet and the skin. Once the liquid is in contact with the skin, the local anesthetic in it is delivered into the skin. The sheet is constructed to have a low enough MVTR to keep the water in the liquid between the sheet and the skin for a time sufficient to deliver the needed amount of the drug for an appropriate duration. The system is maintained in place for a time sufficient to deliver a sufficient amount of the local anesthetic agent for a sufficient length of time to achieve a certain pain reduction or anesthetic effect. An adhesion agent can be incorporated in the liquid to enable the vehicle liquid to serve as a weak “glue” to keep the sheet properly adhered on the skin for the duration of the application period. The adhesion can be strong enough so that the sheet stays on the skin even if the patient moves or changes the position of the body part that contains the skin area. Alternatively, the adhesion agent can be impregnated in the sheet, and dissolves into the vehicle liquid when vehicle liquid is brought into contact with the sheet. The local anesthetic in the liquid may exist in dissolved or undissolved form. The liquid may further comprise a chemical used as a suspension agent to suspend the undissolved local anesthetic particles in the liquid. The liquid can be applied on the target skin area or the sheet in several different ways, including sprayed on the skin or the sheet, brushed on the skin or the sheet; soaked in a sheet of absorbent material (to form a “wet sheet”) which is then applied on the skin or the sheet, or crosslinked into a solidified hydrogel sheet which is then applied on the skin or the sheet. The liquid can have an appropriate viscosity for facilitating the application on the skin or the sheet and the delivery of the drug into the skin. For example, the viscosity can be higher than 100 centipoises, alternatively in the range of 500 to 200,000 centipoise, and alternatively in the range of 1,500 to 50,000 centipoise.

Other embodiments of the current invention are related to a system comprising a liquid comprising water and a sheet comprising a barrier film (whose MVTR is lower than 1,000/m²/24 hour) and bupivacaine, wherein said bupivacaine is distributed co-extensively with said barrier film (either in the barrier film or in another layer of material co-extensive with the barrier film), and wherein said liquid is brought into contact with said bupivacaine within one hour of application of said system on a mammal's skin.

Other embodiments of the current invention are related to a system comprising a liquid comprising water and a sheet comprising a barrier film (whose MVTR is lower than 1,000/m²/24 hour) and diclofenac or a salt of diclofenac, wherein said diclofenac or a salt of diclofenac is distributed co-extensively with said barrier film (either in the barrier film or in another layer of material co-extensive with the barrier film), and wherein said liquid is brought into contact with said diclofenac or a salt of diclofenac within one hour of application of said system on a mammal's skin.

Other embodiments of the current invention are related to a system comprising a liquid comprising water and a sheet comprising a barrier film (whose MVTR is lower than 1,000/m²/24 hour) and ketoprofen, wherein said ketoprofen is distributed co-extensively with said barrier film (either in the barrier film or in another layer of material co-extensive with the barrier film), and wherein said liquid is brought into contact with said ketoprofen within one hour of application of said system on a mammal's skin.

In another embodiment, methods of using the aforementioned two-component drug delivery systems for producing anesthesia in human or other mammalian skin, including tissues under the skin, prior to painful procedures are provided. As described previously, in some of the embodiments, the first component of the system comprises a sheet of a solid and flexible material, and the second component comprises a liquid comprising water. A local anesthetic (such as lidocaine or bupivacaine) is impregnated in the sheet or is in the liquid. A fastening agent can be optionally impregnated in the sheet to fasten the local anesthetic to the sheet (if the local anesthetic is in the sheet). An adhesion agent can be impregnated in the sheet or incorporated in the liquid. The sheet and the liquid are stored separately. To use the system for producing skin anesthesia, the vehicle liquid is applied either on the target skin area or on the sheet, and the sheet is then applied on the target skin area, so that the liquid is between the sheet and the skin. When the vehicle liquid is in contact with the sheet and the skin, the local anesthetic is delivered into the skin. In embodiments in which the local anesthetic is impregnated in the sheet, the local anesthetic is dissolved into the liquid before being delivered into the skin. The system is kept on the skin for sufficient time to deliver a sufficient amount of the local anesthetic agent to produce adequate anesthesia in the tissues. The “sufficient time” depends on factors such as the composition of the vehicle liquid, the local anesthetic, the MVTR of the sheet, the individual's skin permeability to the local anesthetic agent, the depth of the tissue to be anesthetized, and how painful the procedure would be without anesthesia. The “sufficient time” can be as short as 15 minutes, especially for human facial skin or mucosa, but may be 45 minutes to 2 hours or longer. Examples of painful procedures include, but are not limited to, needle injections; laser procedures such as laser tattoo removal, laser spider vein removal, laser hair removal, laser skin resurfacing; and the application of capsaicin-containing formulations on skin, botox or filler injections.

In another embodiment, a method of using the aforementioned two-component drug delivery systems for reducing the pain associated with herpes zoster in the pre-eruptive phase (preherpetic neuralgia), acute eruptive phase, or chronic phase (postherpetic neuralgia), is provided. As described previously, in some of the embodiments, the first component of the system comprises a sheet of a solid and flexible material, and the second component comprises a liquid comprising water. A local anesthetic (such as lidocaine or bupivacaine) is impregnated in the sheet or is in the liquid. A fastening agent can be optionally impregnated in the sheet to fasten the local anesthetic to the sheet (if the local anesthetic is in the sheet). An adhesion agent can be impregnated in the sheet or incorporated in the liquid. The sheet and the liquid are stored separately. To use the system, the vehicle liquid is applied either on the target skin area (the skin area suffering from the aforementioned pain associated with herpes zoster) or on the sheet, and the sheet is then applied on the target skin area, so that the liquid is between the sheet and the skin. When the vehicle liquid is in contact with the sheet and the skin, the local anesthetic is delivered into the skin. In embodiments in which the local anesthetic is impregnated in the sheet, the local anesthetic is dissolved into the liquid before being delivered into the skin. The system is kept on the skin for a sufficient time, such as 30 minutes to 12 hours or longer, to achieve the desired pain reduction effect. Similar systems and methods can also be used to reduce other kinds of neuropathic pain which include but are not limited to pain associated with zoster, diabetes-related nerve damage, neuroma (tumor-induced or trauma-induced); nerve damages caused by viral diseases; nerve compression or pinch, and pain or headache associated with occipital neuralgia.

In another embodiment, a method of using the aforementioned two-component drug delivery systems for reducing musculoskeletal pain is provided. In some of the embodiments, the first component of the system comprises a sheet of a solid and flexible material and has certain barrier property, and the second component comprises a liquid such water. A drug (for example, a local anesthetic such as bupivacaine or lidocaine, or an anti-inflammatory agent such as ketoprofen or diclofenac) is impregnated in the sheet or is in the liquid. The sheet may also comprise a fastening or an adhesion agent (if the drug is in the sheet). The sheet and liquid are stored separately. To use the system, the vehicle liquid is applied either to the sheet or to the target skin area, and the sheet is then applied to the target skin area, so that the vehicle liquid is between the sheet and the liquid. When the vehicle liquid is in contact with the sheet and the skin, the drug is delivered into the skin. (In the embodiments in which the drug is impregnated in the sheet, the drug is dissolved into the liquid before being delivered into the skin). The system is kept on the skin for a pre-determined period of time, for example between one and 12 hours, before being removed. This process may be repeated once or multiple times a day for days or weeks. Musculoskeletal pain includes but is not limited to pain associated with osteoarthritis; rheumatoid arthritis; myofacial pain; carpal tunnel syndrome; complex regional pain syndrome; tennis elbow; soft tissue and bone injuries such as a sprained ankle, knee, shoulder, wrist, elbow, back; and spondylitis. Musculoskeletal pain also includes pain in bones and joints with any or unknown cause, such as neck, knee, spine, or back pain with any or unknown cause. In treating musculoskeletal pain with the system, significant pain reduction may or may not be achievable with a single application of the system. It is possible that multiple applications, wherein each application can last from half an hour to 12 hours or even longer, over a period of hours, days, or even weeks, may be needed before significant pain reduction can be achieved. Similar systems and methods can be used to treat tendonitis and its symptoms including pain. Tendonitis, which may also be spelled “tendinitis,” means the inflammation of a tendon and includes but is not limited to Achilles tendinitis, patellar tendonitis, elbow tendonitis, and wrist tendonitis. In treating tendonitis with the system, significant pain reduction may or may not be achievable with a single application of the system. It is possible that multiple applications, wherein each application can last from half an hour to 12 hours or even longer, over a period of hours, days, or even weeks, may be needed before significant pain reduction can be achieved.

In some of the applications using the embodiments of the current invention, such as aforementioned treatment of musculoskeletal pain, neuropathic pain, and tendonitis, the systems of the current invention may be used with localized heat for achieving deeper penetration of the drug into the tissues. For instance, a lidocaine-impregnated sheet, such as one described in some of the Examples below, can be applied over the knee of a patient suffering from pain associated with arthritis, with the liquid placed between the sheet and the knee skin. A heat generating device, such as a ThermaCare brand air-activated heatwrap, can be placed over the sheet already on the knee. The local heat can increase the skin temperature and may make the drug penetrate deeper into the knee tissues, which may mean better pain relief.

In general, the systems of the current invention separate elements of a dermal drug delivery system into two or more components to gain certain benefits, and provide methods for the components to be joined prior to or during the drug delivery application to deliver the drug at sufficient rates to achieve the desired clinical effect.

Some embodiments of the current invention are related to a sheet for delivering lidocaine into human skin, comprising at least 0.5 mg lidocaine/cm², wherein said sheet's MVTR is lower than 1,000 g/m²/24 hours, and preferably lower than 200 g/m²/24 hours. This sheet can further comprise a lateral diffusion layer which can be a layer of fabric material. The sheet's MVTR property can be provided by a barrier film, such as polyethylene or polyester film. The barrier film and the fabric material layer can be laminated together with heat or adhesive. The sheet can also comprise a fastening agent for fastening the lidocaine and other ingredients onto the sheet. The sheet can further comprise an adhesion agent (such as poly vinyl alcohol) for facilitating the adhering of the sheet, when it is combined with a vehicle liquid, onto the target skin area. This sheet can be free of water. When such a sheet is applied on normal human skin alone or without a vehicle liquid comprising water, it cannot produce anesthesia in said normal human skin within 120 minutes under normal ambient conditions. However, when such a sheet is applied on normal human skin with 25 mg water/cm² between said sheet and said skin, it can produce anesthesia in said normal human skin within 120 minutes under normal ambient conditions. The sheet may further comprise a vasoconstriction agent such as one selected from the group consisting of ephedrine, epinephrine, oxymetazolin, tetrahydrozoline, xylometazoline, phenulphrine, tyramine, naphazoline, caffeine, isoprenaline, pseudoephedrine, orciprenaline, salbutamol, terbutaline.

Some embodiments of the current invention for delivering bupivacaine into human skin are related to a sheet similar to that described in the paragraph above except that the sheet comprises at least 0.5 mg bupivacaine/cm² instead of lidocaine.

Some embodiments of the current invention are related to a sheet for delivering diclofenac into human skin, comprising at least 0.2 mg diclofenac or a salt of diclofenac per cm², wherein said sheet's MVTR is lower than 1,000 g/m²/24 hours, and preferably lower than 200 g/m²/24 hours, This sheet can further comprise a lateral diffusion layer which can be a layer of fabric material, such as woven gauze, non-woven absorbent fabric material, paper, open-cell foam, and cloth. The sheet's MVTR property can be provided by a barrier film, such as polyethylene or polyester film. The barrier film and the fabric material layer can be laminated together with heat or adhesive. The sheet can also comprise a fastening agent for fastening the active drug and other ingredients onto the sheet. The sheet can further comprise an adhesion agent (such as poly vinyl alcohol) for facilitating the adhering of the sheet, when combined with a vehicle liquid, onto the target skin area. This sheet can be free of water.

Some embodiments of the current invention are related to a sheet for delivering ketoprofen into human skin, comprising at least 0.2 mg ketoprofen per cm², wherein said sheet's MVTR is lower than 1,000 g/m²/24 hours, and preferably lower than 200 g/m²/24 hours, This sheet can further comprise a lateral diffusion layer which can be a layer of fabric material, such as woven gauze, non-woven absorbent fabric material, paper, open-cell foam, and cloth. The sheet's MVTR property can be provided by a barrier film, such as polyethylene or polyester film. The barrier film and the fabric material layer can be laminated together with heat or adhesive. The sheet can also comprise a fastening agent for fastening the active drug and other ingredients onto the sheet. The sheet can further comprise an adhesion agent (such as poly vinyl alcohol) for facilitating the adhering of the sheet, when combined with a vehicle liquid, onto the target skin area. This sheet can be free of water.

The Sheet and Liquid Combination system of the current invention has many advantages over other dermal drug delivery dosage forms. Unlike liquid and semisolid dosage forms such as gels, creams, ointments, and solutions, the Sheet and Liquid Combination system provides occlusion over the drug formulation, which can keep the solvent on the skin for longer time and can enhance dermal drug absorption. The sheet can also prevent the drug formulation from being wiped away by objects such as clothing. As a result, much more drug may be delivered into the skin per application. Unlike a reservoir type dermal delivery patch, the sheet of the current invention can be cut into the size and shape for better targeting the treatment skin area. Unlike a drug-in-adhesive type or a hydorgel based dermal drug delivery patch which contains the solvent vehicle for the drug delivery, the Sheet and Liquid Combination system of the current invention does not have the potential problems of drug-solvent reaction, solvent evaporation during storage, or impeding of the drug delivery by the ingredients that are necessary for the required physical or chemical properties of the hydrogel or the drug-in-adhesive matrix. The required physical or chemical properties of the hydrogel or the drug-in-adhesive matrix patches often prevent the patches from using the optimal formulation for delivering the drug. For example, in a drug-in-adhesive patch formulation, the typical adhesive layer, which also serves as the delivery vehicle, may impede the drug permeation significantly due to drug-adhesive interaction, may have the “cold flow” problem, and/or may not have enough volume to hold sufficient amount of the drug or other helpful ingredient(s). For a hydrogel based patch, the hydrogel must be sufficiently adhesive to skin and must be sufficiently cohesive so that it does not fall apart into pieces when is peeled off the skin. These requirements may force the hydrogel formulation to contain certain ingredients and have certain pH that can impede the drug delivery. Indeed, as can be seen in some of the Examples below in comparison with published data, some embodiments of Sheet and Liquid Combination system containing lidocaine can anesthetize intact human skin within 60-75 min, while Lidoderm (a hydrogel based lidocaine patch) cannot anesthetize human skin even after 5 hours for most people, even though both systems contain 5 mg lidocaine per cm², and even though Lidoderm patch contains urea which is a chemical permeation enhancer.

### EXAMPLES

### Example 1

A fabric layer with 5 mg lidocaine base and 5.8 mg PVA per cm² was made with the following method.

Step 1. 1.1 g lidocaine base was dissolved in 10.6 g of a 70% (by volume) isopropyl alcohol solution in a glass jar. 6.3 g of a 20% PVA/80% water solution was added into the jar which was then shaken gently until a clear solution (density about 0.93) was obtained.

Step 2. 16.875 mL of the above solution, in the form of 45×0.375 mL drops dispensed with a multiple pipettor, was dispensed onto 190 cm2 of Dusoft 84148 fabric resting on a release liner.

Step 3. The solution soaked fabric was placed into a 170° F. oven for 66 min for drying. The dried fabric had 5 mg lidocaine base and 5.8 mg PVA per cm2.

Skin test 1. A 2×4 cm piece was cut from the dried sheet and laminated with a 3M 9832 polyurethane tape of the same size, using the tapes adhesive. A piece of 3M 1523 tape (MVTR lower than 100 g/cm2/24 hour) was cut and placed on top of the 3M932 tape to reduce the MVTR of the overall laminated sheet. Fine water drops were sprayed onto the skin on the back of a human hand. The water droplets formed an almost continuous layer of water on the skin. The laminated sheet was placed onto the wet skin. A Kleenex tissue was used to tap on the sheet and surrounding skin area to ensure good skin-sheet contact and remove excess water in the surrounding area. After one hour, the sheet was removed. The skin under the sheet was numb, and was numb for at least another 2.5 hours.

Skin test 2. A 3×4 cm piece was cut from the dried fabric made in Step 3 above, and laminated with a piece of 3M 9832 tape of the same size. Fine water droplets were sprayed onto the forearm skin of a human subject. The water droplets formed an almost continuous layer of water on the skin. The laminated sheet was placed onto the wet skin. A Kleenex tissue was used to tap on the sheet and surrounding skin area to ensure good skin-sheet contact and remove excess water in the surrounding area. A piece of a thin polyethylene film, slightly larger than 3×4 cm, was placed on top of the sheet already on the skin to reduce the evaporation of the water through the sheet. A piece of 3M 9832 tape, larger than the polyethylene film, was placed on top of the film to hold everything under it to the skin. An end of a paper clip was used to test the skin numbness at each time point, with t=0 being the moment the sheet was placed on the skin. The skin was slightly numb at t=60 min. The skin was very numb at t=75 min, 2 hr, 3 hr, 4 hr, 5 hr, 6 hr, 7 hr, 8 hr, 9 hr, 10 hr, and 11 hr. At t=12 hr, the sheet was removed, and the skin was only slightly numb. The numbness in the skin decreased continuously after the sheet was removed, and the skin was not numb at t=13 hr. The skin was a little red when the sheet was removed, but had normal color at t=20 hr.

In this example, the fabric in the sheet is the lateral diffusion layer, PVA in the sheet is the adhesion agent as well as the fastening agent, and the 3M 9832 tape along with the 3M 1523 tape in skin test 1 formed the MVTR control layer. In skin test 2, the two layers of the 3M 9832 tape along with the thin polyethylene film formed the MVTR control layer.

Skin test 3. A 3×10 cm piece was cut from the dried fabric made in Step 3 above, and laminated with a piece of 3M 9830 polyethylene tape (MVTR lower than 100 g/cm2/24 hour) of the same size. Fine water droplets were sprayed onto the skin area just under the patella of a human subject's knee. The water droplets formed an almost continuous layer of water on the skin. The laminated sheet was placed onto the wet skin to cover a horizontal rectangular area just under the patella. A Kleenex tissue was used to tap on the sheet and surrounding skin area to ensure good skin-sheet contact and remove excess water in the surrounding area. An end of a paper clip was used to test the skin numbness at each time point, with t=0 being the moment the sheet was placed on the skin. The skin was numb at t=1 hr, 2 hr, 11 hr, and 12 hr. At t=12 hr, when the sheet still adhered to the skin well, the sheet was removed. In skin test 3, the 3M 9830 tape served as the MVTR control layer.

### Example 2

Three solutions were made, with all the ingredients listed in the table below dissolved in an isopropyl alcohol:water solution.

| | Lidocaine base | Glycerol | PVA |
|---|---|---|---|
| | concentration | concentration | concentration |
| Solution A | 5.84% | 0 | 9.34% |
| Solution B | 5.84% | 2.3% | 9.34% |
| Solution C | 5.84% | 4.7% | 9.34% |

8.75 mL of each of the solutions, in the form of 35×0.25 mL drops dispensed with a multiple pipettor, was dispensed onto 95 cm2 of Dusoft 84148 fabric resting on a release liner.

The solution soaked fabrics were dried at room temperature overnight and in a 170° F. oven for 80 min. The dried fabric pieces (A,B,C, from Solutions A,B,C, respectively) had the following ingredients per cm²:

| | Lidocaine base | Glycerol | PVA |
|---|---|---|---|
| Fabric A | 5 mg/cm² | 0 | 8 mg/cm² |
| Fabric B | 5 mg/cm² | 2 mg/cm² | 8 mg/cm² |
| Fabric C | 5 mg/cm² | 4 mg/cm² | 8 mg/cm² |

After drying, Fabric A was more rigid than Fabric B which was more rigid than Fabric C.

A piece of each of the fabric, about 2×3 cm, was laminated with 3M 9832 polyurethane tape, using the tape's adhesive.

Skin test. To test the effect of the glycerol on skin adhesion and anesthesia, the following experiment was conducted. Fine water droplets were sprayed onto the forearm skin of a human subject. The water droplets formed an almost continuous layer of water on the skin. Each of the laminated sheets was placed onto the wet skin. A Kleenex tissue was used to tap on the sheet and surrounding skin area to ensure good skin-sheet contact and remove excess water in the surrounding area. An end of a paper clip was used to test the skin numbness at each time point, with t=0 being the moment the sheet was placed on the skin. All three sheets were removed from the skin at t=90 min. There was no significant difference in adhesion among the three sheets when they were removed. The following table lists the skin numbness.

| | | Sheet A | Sheet B | Sheet C |
|---|---|---|---|---|
| 60 | min | numb | Numb, but | Slightly numb |
| | | | slightly | |
| | | | shallower than | |
| | | | Sheet A | |
| 90 | min | numb | Numb | Slightly numb |
| 120 | min | numb | Numb | Slightly numb |
| 180 | min | numb | Numb, but | Slightly numb |
| | | | slightly | |
| | | | shallower than | |
| | | | Sheet A | |
| 210 | min | Slightly numb | Almost not numb | Not numb |
| 240 | min | Not numb | Not numb | Not numb |

These results suggest that glycerol can impede the trandermal permeation of lidocaine, but the negative effect from 2 mg/cm² glycerol (Sheet B) is not very significant.

### Example 3

0.28 gram of lidocaine base was added into 7 gram of a 20% poly vinyl alcohol (PVA), 80% water. The two components were stirred well and heated to about 76° C. so that the lidocaine base was melted. Four grams of a viscous solution containing 1.6% Carpobol 981 NF, 0.9% NaOH, and 97.5% water was added into and mixed with the lidocaine-PVA solution mixture. A viscous fluid containing about 2.5% lidocaine, 12.4% PVA, 0.6% Carbopol 981 NF, 0.3% NaOH, and 84.2% water was obtained. The lidocaine in the fluid was not completely dissolved. The undissolved lidocaine, in the form of undissolved solid particles, was suspended in the viscous solution, with the Carbopol 981 NF and PVA serving as the suspension agents. This viscous liquid was the liquid part of the Sheet Liquid Combination system of the current invention.

Separately, a piece of 3M 9830 polyethylene tape was laminated with a piece of Dusoft 84148 rayon polyester blend fabric, using the tape's adhesive. This laminated sheet, with MVTR lower than 100 g/cm²/24 hour, was the sheet part of the sheet-liquid combination system of the current invention.

Skin test. About 0.3 mL of the viscous liquid above was applied to the forearm skin of a human subject. The liquid covered about a 2 cm×3 cm skin area. The 2 cm×3 cm skin area was then covered with a 3 cm×4 cm piece of the laminated sheet, with the fabric side of the sheet in contact with the liquid. The sheet was gently massaged to ensure good contact and even the thickness of the liquid layer underneath it. The sheet adhered to the skin very well mainly because the liquid contained the adhesion agents PVA and Carbopol 981 NF. After one hour, a corner of the sheet was lifted and the skin under it was tested for numbness using the end of a paper clip. The skin was completely numb. The skin surface was moist, indicating that the barrier film of 3M 9830 tape kept the water under the sheet during the application time. The skin under the sheet was tested periodically in the next 11 hours (t=1 hour through t=12 hour) with the same method, and was found to be numb at all of the time points. At t=12 hour, the sheet, which was still adhered well to the skin, was removed from the skin. At that time, the skin under the sheet was numb, and the skin surface was slightly moist.

In this example, the active drug and the adhesion agents were all in the liquid part of the sheet-liquid combination system of the current invention. The sheet served as the barrier to protect the liquid formulation from drying and being wiped away by clothing. The MVTR of the 3M 9830 tape is well below 100 g/m²/24 hour.

It was surprising that the system in the current Example can achieve skin anesthesia as soon as quickly as the EMLA cream (60 min or longer, as reported in published literature) which contains a total of 5% total local anesthetics, and claims using a permeation-enhancing form of the eutectic mixture of lidocaine (2.5%) and prilocalne (2.5%). This example illustrates that with the Sheet and Liquid Combination system of the current invention, desired clinical effect can be achieved with a liquid (in the liquid part of the Sheet and Liquid Combination system) that contains a commonly used local anesthetic, lidocaine, at a commonly used concentration (4% or lower). Therefore, one embodiment of the systems of the current invention involves using a liquid or semisolid (such as gel or very viscous fluid) formulation containing no more than about 4% lidocaine as the only local anesthetic agent, with an occlusive sheet, in methods described in this Example and elsewhere in the current disclosure.

It also should be pointed out that the drug in the liquid part of the current invention does not need to be completely dissolved. One or more suspension agents, such as Carbopol 981 NF or other Carbomers, can be used to suspend the undissolved drug particles in the liquid.

### Example 4

An isopropyl alcohol:water solution containing 9.34% PVA and 1.17% diclofenac sodium is made. 130 mL of the solution is dispensed onto a 50 cm×28 cm piece of a rayon polyester blend fabric (similar to the Derma Sciences Dusoft 84148 fabric) resting on a release liner. The solvents are evaporated off by heating at 200° F. for 20 min. The resulting fabric, containing 1 mg diclofenac sodium and 8 mg PVA per cm², is laminated with 3M 9830 polyethylene tape using the tape's adhesive. The limited sheet is cut into twenty 7 cm×10 cm pieces.

To treat the symptoms associated with osteoarthritis of the knee of a patient, the patient brushes a layer of a viscous aqueous solution (water containing 1.5% Carbopol 981 NF and appropriate concentration of NaOH to adjust the pH to 7.0. The carbopol 981 NF is used as a thickening agent for the solution and an adhesion agent for helping keep the sheet on skin) onto four 7 cm×10 cm skin areas: one just under the patella, one on each side of the patella, and one behind the patella. The patient then places a 7 cm×10 cm piece of the sheet onto each of the four skin areas, with the fabric side of the sheet in contact with the aqueous solution on the skin. He/she then taps the sheets gently to ensure intimate sheet-skin contact. The PVA in the sheet would dissolve into the liquid between the sheet and skin to work as an adhesion agent. The sheets are left on for 8 hours. This treatment process can be repeated twice a day, and for multiple days if needed.

### Example 5

In this Example, similar manufacturing and application methods to those in Example 4 are used, except that the drug is ketoprofen instead of diclofenac sodium.

### Example 6

The Sheet and Liquid Combination systems containing lidocaine, similar to that in Examples 1-3, are used for reducing the pain associated with post herpetic neuralgia. The patient can apply the liquid onto the target skin before applying the sheet on the wet skin, as in Examples 1 and 2. However, because the target skin area may be also suffering from allodynia (hyper-sensitive skin, where even a light touch can cause excruciating pain), an alternative method for applying the liquid involves applying an appropriate quantity of the liquid on the sheet instead of skin. The sheet is then applied on the target skin area, with the side with the vehicle liquid in contact with the skin. More specifically, the liquid is spread on the sheet with a spatula in a quantity of approximately 20 mg/cm². The sheet is then applied onto the skin area suffering from the pain associated with post herpetic neuralgia. The sheet is maintained on the skin area for 8 hours before it is removed. Significant pain reduction would start within about one hour following the commencement of the application, and last throughout the rest of the application time. It would last another 1-2 hours after the sheet is removed at the end of the 8 hour application period.

### Example 7

A sheet containing lidocaine, similar to that in Skin Test 3 of Examples 11 (but large enough in size to cover the target skin area), is used for preventing the pain associated with laser tattoo removal. To use the sheet, a liquid (such as distilled water) is sprayed on the target skin area in a manner similar to that described in Example 1. The sheet is then applied on the liquid-covered target skin area. The sheet is maintained on the skin area for 2 hours before it is removed. The skin area would be anesthetized, and the laser tattoo removal procedure can be performed with minimal or no pain to the patient.

### Example 8

A sheet containing lidocaine, similar to that in Skin Test 3 of Example 1, is used for reducing the pain associated with herpes zoster in the acute eruptive phase. The liquid is spread on the sheet at a quantity of approximately 20 mg/cm². The sheet is then applied onto the skin area suffering from the pain associated with herpes zoster in its eruptive acute phase (with blisters and/or rash). The sheet is maintained on the skin area for 12 hours before it is removed. Significant pain reduction would start within about one hour following the commencement of the application, and would last most or all of the rest of the 12 hour application period.

### Example 9

The system and method for treating herpes zoster in the acute eruptive phase is the same as that in Example 8, except that the drug is bupivacaine instead of lidocaine.

### Example 10

A system, similar to that in Skin Test 3 of Example 1, is used for reducing the pain associated with carpal tunnel syndrome. The liquid, which is contained in a spray bottle, is sprayed on the skin of the carpal tunnel area, at a quantity of approximately 20 mg/cm². The sheet impregnated with lidocaine is then applied onto the skin area and maintained there for 8 hours before it is removed. The application can be repeated at a frequency and for a number of times as adequate for significantly reducing the pain of the individual patient.

### Example 11

A system, similar to that in Example 3, is used for reducing the pain associated with tendonitis of the wrist. The viscous liquid, which contains 2.5% lidocaine, is applied as a continuous layer with a thickness of about 1 mm, on the skin of the wrist suffering from the pain associated with tendonitis. The laminated sheet comprising a 3M 9830 tape layer and a fabric layer, is placed on top of the fluid layer, and is held on the skin by the viscous fluid (mainly due to the adhesion agents PVA and Carbopol 981 NF in the fluid) for 12 hours.

### Example 12

A system and method, similar to that in Example 11, is used to treat the pain associated with patellar tendonitis.

### Example 13

The system and method for treating tendonitis in this Example is similar to that in Examples 11 and 12, except that the viscous liquid contains 1.5% diclofenac soldium instead of 2.5% lidocaine.

### Example 14

The system and method for treating tendonitis in this Example is similar to that in Example 13, except that the viscous liquid contains 1.5% ketoprofen instead of 1.5% diclofenac sodium.

### Example 15

The Sheet and Liquid Combination system in this example have the same components and ingredients as in Example 3, except the sheet has the dimensions of 10 cm×14 cm. About 10 mL of the liquid containing lidocaine is dispensed onto and spread on the fabric side of the sheet to cover the central 8 cm×12 cm of the sheet. The sheet with the liquid is then placed on a human back skin area under which a back pain exists. The sheet is held on the skin for 12 hours by the viscous fluid (mainly due to the adhesion agents PVA and Carbopol 981 NF in the fluid) for treating the back pain.

### Example 16

The system containing lidocaine for reducing the pain or headache associated with occipital neuralgia is similar to that used in Skin Test 3 of Example 1. To use the system, the liquid is spread on the target skin area (typically the target skin area is the skin area over or adjacent to the occipital nerve), at a quantity of approximately 20 mg/cm². The sheet impregnated with lidocaine is then applied onto the skin area and maintained there for 8 hours before it is removed. The application can be repeated at a frequency and for a number of times as adequate for reducing the pain or headache of the individual patient.

### Example 17

The system and method for reducing back pain is similar to that in Example 16, except that the active drug is bupivacaine instead of lidocaine.

### Example 18

This system and method for reducing back pain is similar to that in Example 16, except that the active drug is diclofenac sodium instead of lidocaine.

### Example 19

This system and method for reducing back pain is similar to that in Example 16, except that the active drug is ketoprofen instead of lidocaine.

### Example 20

This system for reducing pain associated with osteoarthritis of the knee (OA of the knee) comprises a sheet comprising a barrier layer and an absorbent fabric layer, and a viscous liquid containing 1% diclofenac soldium, PVA, and water. To use the system, the viscous liquid is applied as a layer on the front, back, and sides of the knee suffering from OA, at a quantity of approximately 30 mg/cm². One or more pieces of the sheet is applied to cover the viscous liquid layer on the skin. The sheet or sheets are maintained on the skin for eight hours before removal. The application can be repeated at a frequency and for a number of times as adequate for reducing the pain of the individual patient.

### Example 21

This system and method in this Example are similar to that in Example 20, except in this case the active drug is ketoprofen and the purpose is to treat pain associated with sprained knee.

### Example 22

This system for reducing pain associated with sprained joints, including sprained ankle, knee, or shoulder, comprises a liquid comprising water and sheets comprising a barrier film and a fabric layer and being impregnated with 1 mg ketoprofen per cm². To use the system, the liquid, at a quantity of approximately 30 mg/cm² is sprayed on the skin area over the sprained joint, and one or more pieces of the sheet are placed on the wet skin. The size and number of the pieces of the skeet used are determined by factors such as the size of the joint and the curvature of the skin area, to maximize comfort and minimize the interference with joint movement and potential of separation of the sheet(s) from the skin. The sheet(s) is maintained there for 8 hours before removal. The application can be repeated at a frequency and for a number of times as adequate for reducing the pain of the individual patient.

### Example 23

In this Example, the systems and methods for reducing back pain, pain associated with OA of the knee, rheumatoid arthritis of the knee, tendonitis of the knee, and sprained joints, including sprained ankle, knee, or shoulder are similar to that used in Examples above except that a ThermaCare brand air-activated heat wrap is applied over the sheet which is already applied on the skin. The local heating can increase the permeability of the skin to the drug and may help drive the drug into deeper tissues, which may mean better clinical results.

### Example 24

In the embodiments in which the drug is in the liquid part of the sheet Liquid Combination system and a portion of the drug exists as undissolved particles, a suspension agent(s) may be needed for suspending the drug particles in the liquid. However, if the adhesion agent is also in the liquid part, the suspension agent may interact with the adhesion agent. As a result, the function of the suspension agent and/or the adhesion agent may be compromised during the storage time of the liquid. For example, the adhesion agent PVA may compromise (reduce) the suspension ability of suspension agent Carbopol 981 (an acrylic polymer). Therefore, in some embodiments of the current invention, the adhesion agent is impregnated in the sheet, and the suspension agent is in the liquid. When the sheet and liquid are combined, the adhesion agent dissolves into the liquid and performs its adhesion task.

In this Example, 2.5 parts of lidocaine base (in the form of particles) were mixed into 97.5 parts of a viscous liquid containing 1.6% Carbopol 981 NF, 0.9% NaOH, and 97.5% water. The mixture was heated to 175° F. for about 30 min, well stirred, let cool to room temperature, and well stirred again. The lidocaine base particles were suspended well in the final viscous liquid.

Separately, a rayon-polyester blend fabric (Dusoft 84148) was impregnated with 2 mg PVA/cm² and laminated to a barrier film (3M9834), using the tape's adhesive.

Skin test. A layer (about 1 mm thick) of the final viscous liquid was applied to the forearm skin of a human subject to cover an area of about 2 cm×3 cm. A piece of the laminated sheet, slightly larger than the 2 cm×3 cm liquid layer on the skin, was placed on top of the liquid layer, with the fabric side in contact with the liquid layer. The sheet immediately adhered well to the liquid and the skin. When the sheet was removed after 75 min, the skin under the sheet was numb. The sheet stayed adhered to the skin very well during the entire 75 min test period.

In this example, the suspension agent Carbopol 981 NF was in the liquid and the adhesion agent PVA was in the sheet. The potential negative interaction between the suspension agent and the adhesion agent during storage was avoided.

It will be appreciated by those having skill in the art that many changes may be made to the details of the above-described embodiments without departing from the underlying principles of the invention. The scope of the present invention should, therefore, be determined only by the following claims.

1. A system for delivering a drug into human skin, comprising:
a sheet comprising a barrier film, wherein said sheet is free of water; and
a liquid comprising water,
wherein one of said sheet and said liquid comprises a drug, wherein said drug is selected from at least one of lidocaine, bupivacaine, diclofenac, or ketoprofen, and wherein said sheet and said liquid are stored separately and are joined within 24 hours of the application of the system on the skin.

2. The system of claim 1,
wherein an adhesion agent exists either in said sheet or said liquid.

3. The system of claim 1, wherein said drug is selected from at least one of lidocaine or bupivacaine, and wherein an application of said sheet on a normal human skin surface with said liquid placed between said sheet and said normal human skin surface is sufficient to achieve anesthesia in said normal human skin within 120 minutes of application under normal ambient conditions.

4. (canceled)

5. (canceled)

6. The system of claim 2, wherein the adhesion agent is impregnated in said sheet.

7. (canceled)

8. The system of claim 1, wherein the MVTR of said barrier film is below about 1,000 g/m²/24 hours.

9. The system of claim 1, wherein said sheet further comprises an adhesion agent which, when the sheet is applied to the skin with the liquid in between the skin and the sheet, is capable of dissolving into said liquid in a sufficient quantity to make the liquid capable of properly adhering said sheet to human skin.

10-12. (canceled)

13. The system of claim 1, wherein said liquid further comprises a suspension agent.

14. (canceled)

15. (canceled)

16. The system of claim 1, further comprising a vasoconstriction agent in said sheet or said liquid.

17. The system of claim 1, wherein at least one part of said sheet or said liquid comprises poly vinyl alcohol.

18. (canceled)

19. The system of claim 1, wherein said sheet further comprises a lateral diffusion layer.

20. The system of claim 1, wherein said sheet further comprises a liquid retention layer.

21. (canceled)

22. The system of claim 1, wherein said system further comprises a device capable of generating heat.

23. A method for anesthetizing human skin, comprising the steps of:
placing an appropriate quantity of a liquid comprising water and a sheet of a material impregnated with a local anesthetic, wherein the local anesthetic is selected from at least one of lidocaine or bupivacaine on a human skin area, and wherein said liquid is placed between said human skin area and said sheet; and
maintaining said sheet on said human skin area for a sufficient time to achieve skin anesthesia, wherein said sheet is constructed to have a sufficiently low MVTR to keep said water between the skin and the sheet for a long enough time to achieve skin anesthesia of normal human skin within 120 minutes of application to the skin under normal ambient conditions.

24-35. (canceled)

36. The method of claim 23, wherein said sheet and said liquid are stored separately and are brought into contact with each other within 24 hours before the application on the human skin area.

37. (canceled)

38. The method of claim 23, wherein said sheet comprises a fabric layer.

39-53. (canceled)

54. A sheet for delivering a drug into human skin, comprising:
a sheet comprising a barrier film and at least 0.5 mg/cm² of a drug, wherein the drug is selected from at least one of lidocaine, bupivacaine, diclofenac, or ketoprofen, wherein said sheet is free of water, and wherein an MVTR of said sheet is lower than 200 g/m²/24 hour.

55. The sheet of claim 54, wherein said sheet further comprises a lateral diffusion layer.

56. The sheet of claim 54, wherein said sheet further comprises an adhesion agent.

57. The sheet of claim 54, wherein said sheet further comprises poly vinyl alcohol.

58-61. (canceled)

62. The system of claim 1, wherein said liquid comprises lidocaine, and wherein a part of said lidocaine exists as undissolved solid particles in said liquid.

63-71. (canceled)

72. The sheet of claim 54, wherein said sheet itself is not adhesive to dry human skin.
